(19) United States
(12) Patent Application Publication
(10) Pub. No.: US 2023/0077014 A1
(43) Pub. Date: Mar. 9, 2023

(54) COMPOSITIONS AND METHODS RELATED TO PHARMACEUTICAL EXCIPIENTS

(71) Applicant: NATURAL EXTRACTION SYSTEMS, LLC, Boulder, CO (US)

(72) Inventor: Douglas G. Metcalf, Boulder, CO (US)

(73) Assignee: NATURAL EXTRACTION SYSTEMS, LLC, Boulder, CO (US)

(21) Appl. No.: 17/797,082

(22) PCT Filed: Feb. 2, 2021
(86) PCT No.: PCT/US2021/016264

§ 371 Date: Aug. 2, 2022

Related U.S. Application Data
(60) Provisional application No. 62/969,618, filed on Feb. 3, 2020.
(60) Provisional application No. 62/971,804, filed on Feb. 7, 2020.
(60) Provisional application No. 63/003,753, filed on Apr. 1, 2020.
(60) Provisional application No. 63/059,825, filed on Jul. 31, 2020.

(51) Int. Cl. A61K 9/08; A61K 47/02; A61K 47/10; A61K 47/22

(52) U.S. Cl. CPC A61K 9/08; A61K 47/02; A61K 47/10; A61K 47/22

(57) ABSTRACT

This patent document describes excipients that allow certain bioactive agents that exist as hydrophobic molecules to be reformulated as anions that display improved hydrophilicity, which can improve the bioavailability and pharmacokinetics of broad classes of pharmaceuticals.

### CROSS-REFERENCE TO RELATED APPLICATIONS

This patent application claims priority to U.S. Provisional Patent Application No. 62/969,618, filed Feb. 3, 2020, U.S. Provisional Patent Application No. 62/971,804, filed Feb. 7, 2020, U.S. Provisional Patent Application No. 63/003,753, filed Apr. 1, 2020, and U.S. Provisional Patent Application No. 63/059,825, filed Jul. 31, 2020, each of which is incorporated by reference in its entirety.

### BACKGROUND

The “Rule of Five” states that druglike molecules generally have octanol-water partition coefficients of no greater than 5. A generally-applicable strategy to overcome this feature of the Rule of Five is desirable.

### SUMMARY

An octanol-water partition coefficient of greater than 5 limits solubility in bodily fluids, which limits bioavailability. Many classes of hydrophobic bioactive molecules can be converted into anions that display improved octanol-water partition coefficients using simple acid/base chemistry. Such anions convert back into their parent bioactive molecules upon administration to a subject. Various aspects of this patent document relate to the discovery that the anionic form of a bioactive agent can overcome the octanol-water partition coefficient limitation set forth in the Rule of Five.

Novel aqueous compositions that utilize the inventive chemistry in relation to specific classes of bioactive agents are disclosed, for example, in U.S. Pat. Nos. 10,555,914, 10,609,944. This patent document discloses novel features of excipients that are generally compatible with broad classes of bioactive agents that can overcome the octanol-water partition coefficient limitation set forth in the Rule of Five by conversion into anionic formats.

### DETAILED DESCRIPTION

Various aspects of this patent document relate to a composition, comprising a solvent, a Brønsted base, a cation, an anion, and a molecule, wherein the solvent has the chemical formula CₓH_{y}O_{z}; each carbon atom of the solvent is an unsaturated carbon atom; x is an integer of at least 2 and no greater than 7; either the solvent has the chemical formula C₆H₁₂O₆, or y equals 2× plus 2; the solvent comprises z hydroxyl group(s); each hydroxyl group of the solvent is covalently bound to a different carbon atom of the solvent; z is an integer of at least 1 and no greater than x; the Brønsted base is a conjugate base of the solvent; the Brønsted base has the chemical formula CₓH_{w}O_{z}¹⁻; the Brønsted base comprises 1 oxide group; the Brønsted base comprises z minus 1 hydroxyl group(s); w equals y minus 1; the cation is either a metal cation or an ammonium cation; the anion is a conjugate base of the molecule; the molecule has an acid dissociation constant in water of at least 50 femtomolar and no greater than 50 nanomolar for conversion of the molecule into the anion; the composition comprises a liquid phase that is miscible with water; the liquid phase has a molar concentration of the solvent, which is at least 5 molar and no greater than 17 molar; the Brønsted base, the cation, the anion, the molecule, and the hydroxide are solutes that are dissolved in the liquid phase; the liquid phase has a molar concentration of the Brønsted base that is dissolved in the liquid phase, which is at least 1 nanomolar and no greater than 1 molar; the molar concentration of the solvent in the liquid phase is at least 10 times greater than the molar concentration of the Brønsted base that is dissolved in the liquid phase; the liquid phase has a molar concentration of the cation that is dissolved in the liquid phase, which is at least 10 nanomolar and no greater than 1 molar; the liquid phase has a molar concentration of the anion that is dissolved in the liquid phase, which is at least 10 nanomolar and no greater than 1 molar; and the liquid phase has a molar concentration of the molecule that is dissolved in the liquid phase, which is less than the molar concentration of the anion that is dissolved in the liquid phase.

“Comprise” refers to an open set such that a composition that comprises a solvent, a Brønsted base, a cation, an anion, and a molecule can also comprise the anionic form of a bioactive agent.

“Dissolved” refers to a chemical species that is solvated in a liquid phase, for example, by a solvent; a chemical species that is present within a phase that is dispersed within a liquid phase, such as the dispersed phase of an emulsion, is not dissolved in the liquid phase; a chemical species that is non-covalently bound to any chemical species that is a solid in the absence of a solvent, such as a cyclodextrin, is not dissolved.

“Ammonium cation” refers to either ammonium (“NH₄+”) or an aminium cation.

In some specific embodiments, the solvent has the chemical formula C₂H₆O, C₃H₈O, C₃H₈O₂, C₃H₈O₃, C₄H₁₀O, C₄H₁₀O₂, C₄H₁₀O₃, C₄H₁₀O₄, C₅H₁₂O, C₅H₁₂O₂, C₅H₁₂O₃, C₅H₁₂O₄, C₅H₁₂O₅, C₆H₁₂O₆, C₆H₁₄O, C₆H₁₄O₂, C₆H₁₄O₃, C₆H₁₄O₄, C₆H₁₄O₅, C₆H₁₄O₆, C₇H₁₆O, C₇H₁₆O₂, C₇H₁₆O₃, C₇H₁₆O₄, C₇H₁₆O₅, C₇H₁₆O₆, or C₇H₁₆O₇. In some very specific embodiments, the solvent has the chemical formula C₂H₆O, C₃H₈O₂, C₃H₈O₃, C₄H₁₀O₂, C₄H₁₀O₄, C₅H₁₂O₅, C₆H₁₂O₆, C₆H₁₄O₆, or C₇H₁₆O₇.

In some very specific embodiments, the solvent is 1,2-propanediol; 1,3-propanediol; 1,2,3-propanetriol; 1,3-butanediol; 1,4-butanediol; 2,3-butanediol; butane-1,2,3,4-tetrol; pentane-1,2,3,4,5-pentol; cyclohexane-1,2,3,4,5,6-hexol; hexane-1,2,3,4,5,6-hexol; or heptane-1,2,3,4,5,6,7-heptol.

In some specific embodiments, the liquid phase has a molar concentration of the solvent, which is at least 5 molar and no greater than 14 molar.

In some specific embodiments, the Brønsted base has the chemical formula C₂H₅O¹⁻, C₃H₇O¹⁻, C₃H₇O₂¹⁻, C₃H₇O₃¹⁻, C₄H₉O¹⁻, C₄H₉O₂¹⁻, C₄H₉O₃¹⁻, C₄H₉O₄¹⁻, C₅H₁₁O¹⁻, C₅H₁₁O₂¹⁻, C₅H₁₁O₃¹⁻, C₅H₁₁O₄¹⁻, C₅H₁₁O₅¹⁻, C₆H₁₁O₆¹⁻, C₆H₁₃O¹⁻, C₆H₁₃O₂¹⁻, C₆H₁₃O₃¹⁻, C₆H₁₃O₄¹⁻, C₆H₁₃O₅¹⁻, C₆H₁₃O₆¹⁻, C₇H₁₅O¹⁻, C₇H₁₅O₂¹⁻, C₇H₁₅O₃¹⁻, C₇H₁₅O₄¹⁻, C₇H₁₅O₅¹⁻, C₇H₁₅O₆¹⁻, or C₇H₁₅O₇¹. In some very specific embodiments, the Brønsted base has the chemical formula C₂H₅O¹⁻, C₃H₇O₂¹⁻, C₃H₇O₃¹⁻, C₄H₉O₂¹⁻, C₄H₉O₄¹⁻, C₅H₁₁O₅¹⁻, C₆H₁₁O₆¹⁻, C₆H₁₃O₆¹⁻, or C₇H₁₅O₇¹⁻.

In some embodiments, the Brønsted base is ethoxide; propane-1-oxide; 1-hydroxypropane-2-oxide; 2-hydroxypropane-1-oxide; 3-hydroxypropane-1-oxide; 1,3-dihydroxypropane-2-oxide; 2,3-dihydroxypropane-1-oxide; butane-1-oxide; butane-2-oxide; 1-hydroxybutane-2-oxide; 2-hydroxybutane-1-oxide; 3-hydroxybutane-1-oxide; 3-hydroxybutane-2-oxide; 4-hydroxybutane-1-oxide; 4-hydroxybutane-2-oxide; 1,3,4-trihydroxybutane-2-oxide; 2,3,4-trihydroxybutane-1-oxide; pentane-1-oxide; pentane-2-oxide; pentane-3-oxide; 1,2,4,5-tetrahydroxypentane-3-oxide; 1,3,4,5-tetrahydroxypentane-2-oxide; 2,3,4,5-tetrahydroxypentane-1-oxide; 1,2,4,5,6-pentahydroxyhexane-3-oxide; 1,3,4,5,6-pentahydroxyhexane-2-oxide; 2,3,4,5,6-pentahydroxyhexane-1-oxide; 2,3,4,5,6-pentahydroxycyclohexane-1-oxide; 1,2,3,5,6,7-hexahydroxyheptane-4-oxide; 1,2,4,5,6,7-hexahydroxyheptane-3-oxide; 1,3,4,5,6,7-hexahydroxyheptane-2-oxide; or 2,3,4,5,6,7-hexahydroxyheptane-1-oxide. In some specific embodiments, the Brønsted base is ethoxide; 1-hydroxypropane-2-oxide; 2-hydroxypropane-1-oxide; 3-hydroxypropane-1-oxide; 1,3-dihydroxypropane-2-oxide; 2,3-dihydroxypropane-1-oxide; 1,3,4-trihydroxybutane-2-oxide; 2,3,4-trihydroxybutane-1-oxide; 1,2,4,5-tetrahydroxypentane-3-oxide; 1,3,4,5-tetrahydroxypentane-2-oxide; 2,3,4,5-tetrahydroxypentane-1-oxide; 1,2,4,5,6-pentahydroxyhexane-3-oxide; 1,3,4,5,6-pentahydroxyhexane-2-oxide; 2,3,4,5,6-pentahydroxyhexane-1-oxide; 2,3,4,5,6-pentahydroxycyclohexane-1-oxide; 1,2,3,5,6,7-hexahydroxyheptane-4-oxide; 1,2,4,5,6,7-hexahydroxyheptane-3-oxide; 1,3,4,5,6,7-hexahydroxyheptane-2-oxide; or 2,3,4,5,6,7-hexahydroxyheptane-1-oxide. In some very specific embodiments, the Brønsted base is 1-hydroxypropane-2-oxide; 2-hydroxypropane-1-oxide; 3-hydroxypropane-1-oxide; 1,3-dihydroxypropane-2-oxide; or 2,3-dihydroxypropane-1-oxide.

In some specific embodiments, the molar concentration of the Brønsted base that is dissolved in the liquid phase is at least 10 nanomolar and no greater than 100 millimolar.

In some specific embodiments, the molar concentration of the solvent in the liquid phase is at least 100 times greater than the molar concentration of the Brønsted base that is dissolved in the liquid phase.

In some embodiments, the composition comprises water and hydroxide; the hydroxide is a solute that is dissolved in the liquid phase; the liquid phase has a molar concentration of the hydroxide that is dissolved in the liquid phase; and the liquid phase has a molar concentration of water, which is at least 10 times and no more than 1 billion times greater than the molar concentration of the hydroxide that is a dissolved in the liquid phase. In some specific embodiments, the liquid phase has a molar concentration of water, which is at least 100 times and no more than 100 million times greater than the molar concentration of the hydroxide that is a dissolved in the liquid phase. In some very specific embodiments, the liquid phase has a molar concentration of water, which is at least 1000 times and no more than 10 million times greater than the molar concentration of the hydroxide that is a dissolved in the liquid phase.

In some embodiments, the composition comprises ethoxide.

In some specific embodiments, the cation in is a metal cation. In some very specific embodiments, the cation is a metal cation, and the metal cation is lithium cation (“Li+”); sodium cation (“Na+”); potassium cation (“K+”); magnesium cation (“Mg++”); calcium cation (“Ca++”); zinc cation (“Zn++”); manganese cation (“Mn++”); iron (II) cation (“Fe++”); iron (III) cation (“Fe+++”); copper (I) cation (“Cu+”); or copper (II) cation (“Cu++”).

In some specific embodiments, the cation is a metal cation, and the metal cation is potassium cation or sodium cation. In some very specific embodiments, the cation is a metal cation, and the metal cation is potassium cation. In some very specific embodiments, the cation is a metal cation, and the metal cation is sodium cation.

In some specific embodiments, the cation is an ammonium cation. In some very specific embodiments, the cation is an ammonium cation, and the ammonium cation is ammonium; protonated ethanolamine; choline; protonated lysine; protonated arginine; or protonated sphingosine.

In some specific embodiments, the molar concentration of the cation that is dissolved in the liquid phase is at least 100 nanomolar and no greater than 100 millimolar.

In some embodiments, the molecule has the chemical formula C_{(MC)}H_{(MH)}X_{(MX)}N_{(MN)}O_{(MO)}S_{(MS)}; the anion has the chemical formula C_{(AC)}H_{(AH)}X_{(AX)}N_{(AN)}O_{(AO)}S_{(AS)}¹⁻; MC is an integer from 6 to 32; MH is an integer from 6 to 50; MH is both at least half MC and no more than 1.75 times greater than MC; X is a halogen selected from F, Cl, Br, and I; MX is either 0, 1, or 2; MN is either 0, 1, or 2; MO is an integer from 1 to 12; MC is at least 2 times greater than MO; MS is either 0 or 1; AC equals MC; AH equals MH minus 1; AX equals MX; AN equals MN; AO equals MO; and AS equals MS. In some specific embodiments, the molecule has the chemical formula C_{(MC)}H(MH)N_{(MN)}O_{(MO)}; and the anion has the chemical formula C_{(AC)}H_{(AH)}N_{(AN)}O_{(AO)}¹⁻. In some specific embodiments, the molecule has the chemical formula C_{(MC)}H_{(MH)}O_{(MO)}S_{(MS)}; and the anion has the chemical formula C_{(AC)}H_{(AH)}O_{(AO)}S_{(AS)}¹⁻. In some specific embodiments, the molecule has the chemical formula C_{(MC)}H_{(MH)}X_{(MX)}O_{(MO)}; and the anion has the chemical formula C_{(AC)}H_{(AH)}X_{(AX)}O_{(AO)}¹⁻. In some very specific embodiments, the molecule has the chemical formula C_{(MC)}H_{(MH)}O_{(MO)}; and the anion has the chemical formula C_{(AC)}H_{(AH)}O_{(AO)}¹⁻.

The nature of the molecule and the anion is not particularly limiting so long as the molecule has an acid dissociation constant in water of at least 50 femtomolar and no greater than 50 nanomolar for conversion of the molecule into the anion. A molecule that comprises a hydroxyl group that is bound to an unsaturated carbon atom typically has an acid dissociation constant in water of at least 50 femtomolar and no greater than 50 nanomolar for conversion of the molecule into the anion provided that the hydroxyl group is the most acidic functional group of the molecule. Examples of such molecules include numerous natural products such as eugenol, thymol, and carvacrol.

In some specific embodiments, the molecule has an acid dissociation constant in water of at least 100 femtomolar and no greater than 10 nanomolar for conversion of the molecule into the anion. In some very specific embodiments, the molecule has an acid dissociation constant in water of at least 5 picomolar and no greater than 5 nanomolar for conversion of the molecule into the anion.

In all embodiments, the molecule and anion buffer the pH of the liquid phase such that the bioactive agent exists primarily in an anionic form. In some very specific preferred embodiments, the molecule is the molecular form of the bioactive agent and the anion is the anionic form of the bioactive agent, for example, such that the anionic and molecular forms of the bioactive agent act as their own buffer. In such embodiments, the anionic form of the bioactive agent is typically present at a therapeutically effective concentration, and the molecular form of the bioactive agent is typically present at a concentration that is less than any known therapeutically effective concentrations for the molecular form.

In some specific embodiments, the molar concentration of the anion that is dissolved in the liquid phase is at least 100 nanomolar and no greater than 100 millimolar.

Various aspects of this patent document relate to a composition described anywhere in this patent document for use as a medicament.

Various aspects of this patent document relate to a method to administer a bioactive agent to a subject, comprising providing a composition described anywhere in this patent document, and administering the composition to the subject.

In some embodiments, administering is oral, sublingual, sublabial, buccal, rectal, intranasal, inhalational, transmucosal, topical, transdermal, intravenous, intramuscular, subcutaneous, intradermal, intraocular, parenteral, intrathecal, intralesional, or intratumoral administering. In some specific embodiments, administering is oral, sublingual, or buccal administering.

In some specific embodiments, the composition is formulated to allow the conversion of an anionic form of the bioactive agent into a molecular form of the bioactive agent. In some very specific embodiments, the composition is formulated to allow the conversion of the anionic form of the bioactive agent into the molecular form of the bioactive agent ex vivo prior to administering the composition to the subject. In some very specific embodiments, the composition is formulated to allow the conversion of the anionic form of the bioactive agent into the molecular form of the bioactive agent in situ subsequent to administering the composition to the subject.

In some specific embodiments, administering comprises topical administration. In some very specific embodiments, the composition is formulated for topical administration, and topical administration results in conversion of an anionic form of the bioactive agent into a molecular form of the bioactive agent.

In some specific embodiments, administering comprises oral administration. In some very specific preferred embodiments, the composition is formulated for oral administration, and the composition is formulated to allow the conversion of an anionic form of the bioactive agent into a molecular form of the bioactive agent before the bioactive agent reaches the stomach of the subject to allow absorption of the bioactive agent by the epithelial lining of the gastrointestinal tract between the lips and the stomach, excluding the stomach and the outer surfaces of the lips, and including the esophagus and the inner surfaces of the lips.

In some specific embodiments, the subject is a rodent, lagomorph, feline, canine, porcine, ovine, caprine, lama, vicugna, bovine, equine, or primate. In some very specific embodiments, the subject is human.

What is claimed is:

1. A composition, comprising a solvent, a Brønsted base, a cation, an anion, a molecule, water, and hydroxide, wherein:
the solvent has the chemical formula CₓH_{y}O_{z}, wherein each carbon atom of the solvent is an unsaturated carbon atom; x is an integer of at least 2 and no greater than 7; either the solvent has the chemical formula C₆H₁₂O₆, or y equals 2× plus 2; the solvent comprises z hydroxyl group(s); each hydroxyl group of the solvent is covalently bound to a different carbon atom of the solvent; and z is an integer of at least 1 and no greater than x;
the Brønsted base is a conjugate base of the solvent;
the Brønsted base has the chemical formula CₓH_{w}O_{z}¹⁻, wherein the Brønsted base comprises 1 oxide group; the Brønsted base comprises z minus 1 hydroxyl group(s); and w equals y minus 1;
the cation is either a metal cation or an ammonium cation;
the anion is a conjugate base of the molecule;
the molecule has an acid dissociation constant in water of at least 50 femtomolar and no greater than 50 nanomolar for conversion of the molecule into the anion;
the composition comprises a liquid phase that is miscible with water;
the liquid phase has a molar concentration of the solvent, which is at least 5 molar and no greater than 17 molar;
the Brønsted base, the cation, the anion, the molecule, and the hydroxide are solutes that are dissolved in the liquid phase;
the liquid phase has a molar concentration of the Brønsted base that is dissolved in the liquid phase, which is at least 1 nanomolar and no greater than 1 molar;
the molar concentration of the solvent in the liquid phase is at least 10 times greater than the molar concentration of the Brønsted base that is dissolved in the liquid phase;
the liquid phase has a molar concentration of the cation that is dissolved in the liquid phase, which is at least 10 nanomolar and no greater than 1 molar;
the liquid phase has a molar concentration of the anion that is dissolved in the liquid phase, which is at least 10 nanomolar and no greater than 1 molar;
the liquid phase has a molar concentration of the molecule that is dissolved in the liquid phase, which is less than the molar concentration of the anion that is dissolved in the liquid phase;
the liquid phase has a molar concentration of the hydroxide that is dissolved in the liquid phase; and
the liquid phase has a molar concentration of water, which is at least 10 times and no more than 1 billion times greater than the molar concentration of the hydroxide that is a dissolved in the liquid phase.

2. The composition of claim 1, wherein the molecule has the chemical formula C₂H₆O, C₃H₈O₂, C₃H₈O₃, C₄H₁₀O₂, C₄H₁₀O₄, C₅H₁₂O₅, C₆H₁₂O₆, C₆H₁₄O₆, or C₇H₁₆O₇.

3. The composition of claim 1 or 2, wherein the molecule is 1,2-propanediol; 1,3-propanediol; 1,2,3-propanetriol; 1,3-butanediol; 1,4-butanediol; 2,3-butanediol; butane-1,2,3,4-tetrol; pentane-1,2,3,4,5-pentol; cyclohexane-1,2,3,4,5,6-hexol; hexane-1,2,3,4,5,6-hexol; or heptane-1,2,3,4,5,6,7-heptol.

4. The composition of any one of claims 1-3, wherein the Brønsted base is 1-hydroxypropane-2-oxide; 2-hydroxypropane-1-oxide; 3-hydroxypropane-1-oxide; 1,3-dihydroxypropane-2-oxide; or 2,3-dihydroxypropane-1-oxide.

5. A composition, comprising a solvent, a Brønsted base, a cation, an anion, and a molecule, wherein:
the solvent is 1,2-propanediol;
the Brønsted base is 1-hydroxypropane-2-oxide or 2-hydroxypropane-1-oxide;
the anion is a conjugate base of the molecule;
the molecule has an acid dissociation constant in water of at least 50 femtomolar and no greater than 50 nanomolar for conversion of the molecule into the anion;
the composition comprises a liquid phase that is miscible with water;
the liquid phase has a molar concentration of the solvent, which is at least 5 molar and no greater than 14 molar;
the Brønsted base, the cation, the anion, and the molecule are solutes that are dissolved in the liquid phase;
the liquid phase has a molar concentration of the Brønsted base that is dissolved in the liquid phase, which is at least 1 nanomolar and no greater than 1 molar;
the molar concentration of the solvent in the liquid phase is at least 10 times greater than the molar concentration of the Brønsted base that is dissolved in the liquid phase;
the liquid phase has a molar concentration of the cation that is dissolved in the liquid phase, which is at least 10 nanomolar and no greater than 1 molar;
the liquid phase has a molar concentration of the anion that is dissolved in the liquid phase, which is at least 10 nanomolar and no greater than 1 molar; and
the liquid phase has a molar concentration of the molecule that is dissolved in the liquid phase, which is less than the molar concentration of the anion that is dissolved in the liquid phase.

6. A composition, comprising a solvent, a Brønsted base, a cation, an anion, and a molecule, wherein:
the solvent is 1,3-propanediol;
the Brønsted base is 3-hydroxypropane-1-oxide;
the anion is a conjugate base of the molecule;
the molecule has an acid dissociation constant in water of at least 50 femtomolar and no greater than 50 nanomolar for conversion of the molecule into the anion;
the composition comprises a liquid phase that is miscible with water;
the liquid phase has a molar concentration of the solvent, which is at least 5 molar and no greater than 14 molar;
the Brønsted base, the cation, the anion, the molecule, and the hydroxide are solutes that are dissolved in the liquid phase;
the liquid phase has a molar concentration of the Brønsted base that is dissolved in the liquid phase, which is at least 1 nanomolar and no greater than 1 molar;
the molar concentration of the solvent in the liquid phase is at least 10 times greater than the molar concentration of the Brønsted base that is dissolved in the liquid phase;
the liquid phase has a molar concentration of the cation that is dissolved in the liquid phase, which is at least 10 nanomolar and no greater than 1 molar;
the liquid phase has a molar concentration of the anion that is dissolved in the liquid phase, which is at least 10 nanomolar and no greater than 1 molar; and
the liquid phase has a molar concentration of the molecule that is dissolved in the liquid phase, which is less than the molar concentration of the anion that is dissolved in the liquid phase.

7. A composition, comprising a solvent, a Brønsted base, a cation, an anion, and a molecule, wherein:
the solvent is 1,2,3-propanetriol;
the Brønsted base is 1,3-dihydroxypropane-2-oxide or 2,3-dihydroxypropane-1-oxide;
the anion is a conjugate base of the molecule;
the molecule has an acid dissociation constant in water of at least 50 femtomolar and no greater than 50 nanomolar for conversion of the molecule into the anion;
the composition comprises a liquid phase that is miscible with water;
the liquid phase has a molar concentration of the solvent, which is at least 5 molar and no greater than 14 molar;
the Brønsted base, the cation, the anion, the molecule, and the hydroxide are solutes that are dissolved in the liquid phase;
the liquid phase has a molar concentration of the Brønsted base that is dissolved in the liquid phase, which is at least 1 nanomolar and no greater than 1 molar;
the molar concentration of the solvent in the liquid phase is at least 10 times greater than the molar concentration of the Brønsted base that is dissolved in the liquid phase;
the liquid phase has a molar concentration of the cation that is dissolved in the liquid phase, which is at least 10 nanomolar and no greater than 1 molar;
the liquid phase has a molar concentration of the anion that is dissolved in the liquid phase, which is at least 10 nanomolar and no greater than 1 molar; and
the liquid phase has a molar concentration of the molecule that is dissolved in the liquid phase, which is less than the molar concentration of the anion that is dissolved in the liquid phase.

8. A composition, comprising a solvent, a Brønsted base, a cation, an anion, and a molecule, wherein:
the solvent is butane-1,2,3,4-tetrol;
the Brønsted base is 1,3,4-trihydroxybutane-2-oxide or 2,3,4-trihydroxybutane-1-oxide;
the anion is a conjugate base of the molecule;
the molecule has an acid dissociation constant in water of at least 50 femtomolar and no greater than 50 nanomolar for conversion of the molecule into the anion;
the composition comprises a liquid phase that is miscible with water;
the liquid phase has a molar concentration of the solvent, which is at least 5 molar and no greater than 14 molar;
the Brønsted base, the cation, the anion, the molecule, and the hydroxide are solutes that are dissolved in the liquid phase;
the liquid phase has a molar concentration of the Brønsted base that is dissolved in the liquid phase, which is at least 1 nanomolar and no greater than 1 molar;
the molar concentration of the solvent in the liquid phase is at least 10 times greater than the molar concentration of the Brønsted base that is dissolved in the liquid phase;
the liquid phase has a molar concentration of the cation that is dissolved in the liquid phase, which is at least 10 nanomolar and no greater than 1 molar;
the liquid phase has a molar concentration of the anion that is dissolved in the liquid phase, which is at least 10 nanomolar and no greater than 1 molar; and
the liquid phase has a molar concentration of the molecule that is dissolved in the liquid phase, which is less than the molar concentration of the anion that is dissolved in the liquid phase.

9. A composition, comprising a solvent, a Brønsted base, a cation, an anion, and a molecule, wherein:
the solvent is pentane-1,2,3,4,5-pentol;
the Brønsted base is 1,2,4,5-tetrahydroxypentane-3-oxide, 1,3,4,5-tetrahydroxypentane-2-oxide, or 2,3,4,5-tetrahydroxypentane-1-oxide;
the anion is a conjugate base of the molecule;
the molecule has an acid dissociation constant in water of at least 50 femtomolar and no greater than 50 nanomolar for conversion of the molecule into the anion;
the composition comprises a liquid phase that is miscible with water;
the liquid phase has a molar concentration of the solvent, which is at least 5 molar and no greater than 14 molar;
the Brønsted base, the cation, the anion, the molecule, and the hydroxide are solutes that are dissolved in the liquid phase;
the liquid phase has a molar concentration of the Brønsted base that is dissolved in the liquid phase, which is at least 1 nanomolar and no greater than 1 molar;
the molar concentration of the solvent in the liquid phase is at least 10 times greater than the molar concentration of the Brønsted base that is dissolved in the liquid phase;
the liquid phase has a molar concentration of the cation that is dissolved in the liquid phase, which is at least 10 nanomolar and no greater than 1 molar;
the liquid phase has a molar concentration of the anion that is dissolved in the liquid phase, which is at least 10 nanomolar and no greater than 1 molar; and
the liquid phase has a molar concentration of the molecule that is dissolved in the liquid phase, which is less than the molar concentration of the anion that is dissolved in the liquid phase.

10. A composition, comprising a solvent, a Brønsted base, a cation, an anion, and a molecule, wherein:
the solvent is cyclohexane-1,2,3,4,5,6-hexol;
the Brønsted base is 2,3,4,5,6-pentahydroxycyclohexane-1-oxide;
the anion is a conjugate base of the molecule;
the molecule has an acid dissociation constant in water of at least 50 femtomolar and no greater than 50 nanomolar for conversion of the molecule into the anion;
the composition comprises a liquid phase that is miscible with water;
the liquid phase has a molar concentration of the solvent, which is at least 5 molar and no greater than 14 molar;
the Brønsted base, the cation, the anion, the molecule, and the hydroxide are solutes that are dissolved in the liquid phase;
the liquid phase has a molar concentration of the Brønsted base that is dissolved in the liquid phase, which is at least 1 nanomolar and no greater than 1 molar;
the molar concentration of the solvent in the liquid phase is at least 10 times greater than the molar concentration of the Brønsted base that is dissolved in the liquid phase;
the liquid phase has a molar concentration of the cation that is dissolved in the liquid phase, which is at least 10 nanomolar and no greater than 1 molar;
the liquid phase has a molar concentration of the anion that is dissolved in the liquid phase, which is at least 10 nanomolar and no greater than 1 molar; and
the liquid phase has a molar concentration of the molecule that is dissolved in the liquid phase, which is less than the molar concentration of the anion that is dissolved in the liquid phase.

11. A composition, comprising a solvent, a Brønsted base, a cation, an anion, and a molecule, wherein:
the solvent is hexane-1,2,3,4,5,6-hexol;
the Brønsted base is 1,2,4,5,6-pentahydroxyhexane-3-oxide, 1,3,4,5,6-pentahydroxyhexane-2-oxide, or 2,3,4,5,6-pentahydroxyhexane-1-oxide;
the anion is a conjugate base of the molecule;
the molecule has an acid dissociation constant in water of at least 50 femtomolar and no greater than 50 nanomolar for conversion of the molecule into the anion;
the composition comprises a liquid phase that is miscible with water;
the liquid phase has a molar concentration of the solvent, which is at least 5 molar and no greater than 14 molar;
the Brønsted base, the cation, the anion, the molecule, and the hydroxide are solutes that are dissolved in the liquid phase;
the liquid phase has a molar concentration of the Brønsted base that is dissolved in the liquid phase, which is at least 1 nanomolar and no greater than 1 molar;
the molar concentration of the solvent in the liquid phase is at least 10 times greater than the molar concentration of the Brønsted base that is dissolved in the liquid phase;
the liquid phase has a molar concentration of the cation that is dissolved in the liquid phase, which is at least 10 nanomolar and no greater than 1 molar;
the liquid phase has a molar concentration of the anion that is dissolved in the liquid phase, which is at least 10 nanomolar and no greater than 1 molar; and
the liquid phase has a molar concentration of the molecule that is dissolved in the liquid phase, which is less than the molar concentration of the anion that is dissolved in the liquid phase.

12. A composition, comprising a solvent, a Brønsted base, a cation, an anion, and a molecule, wherein:
the solvent is heptane-1,2,3,4,5,6,7-heptol;
the Brønsted base is 1,2,3,5,6,7-hexahydroxyheptane-4-oxide, 1,2,4,5,6,7-hexahydroxyheptane-3-oxide, 1,3,4,5,6,7-hexahydroxyheptane-2-oxide, or 2,3,4,5,6,7-hexahydroxyheptane-1-oxide;
the anion is a conjugate base of the molecule;
the molecule has an acid dissociation constant in water of at least 50 femtomolar and no greater than 50 nanomolar for conversion of the molecule into the anion;
the composition comprises a liquid phase that is miscible with water;
the liquid phase has a molar concentration of the solvent, which is at least 5 molar and no greater than 14 molar;
the Brønsted base, the cation, the anion, the molecule, and the hydroxide are solutes that are dissolved in the liquid phase;
the liquid phase has a molar concentration of the Brønsted base that is dissolved in the liquid phase, which is at least 1 nanomolar and no greater than 1 molar;
the molar concentration of the solvent in the liquid phase is at least 10 times greater than the molar concentration of the Brønsted base that is dissolved in the liquid phase;
the liquid phase has a molar concentration of the cation that is dissolved in the liquid phase, which is at least 10 nanomolar and no greater than 1 molar;
the liquid phase has a molar concentration of the anion that is dissolved in the liquid phase, which is at least 10 nanomolar and no greater than 1 molar; and
the liquid phase has a molar concentration of the molecule that is dissolved in the liquid phase, which is less than the molar concentration of the anion that is dissolved in the liquid phase.

13. The composition of any one of claims 5-12, comprising water and hydroxide, wherein the hydroxide is a solute that is dissolved in the liquid phase; the liquid phase has a molar concentration of the hydroxide that is dissolved in the liquid phase; and the liquid phase has a molar concentration of water, which is at least 10 times and no more than 1 billion times greater than the molar concentration of the hydroxide that is a dissolved in the liquid phase.

14. The composition of any one of claims 1-13, comprising ethoxide.

15. The composition of any one of claims 1-14, wherein the cation is lithium cation (“Li+”); sodium cation (“Na+”); potassium cation (“K+”); magnesium cation (“Mg++”); calcium cation (“Ca++”); zinc cation (“Zn++”); manganese cation (“Mn++”); iron (II) cation (“Fe++”); iron (III) cation (“Fe+++”); copper (I) cation (“Cu+”); copper (II) cation (“Cu++”); ammonium (“NH₄+”); protonated ethanolamine; choline; protonated lysine; protonated arginine; or protonated sphingosine.

16. The composition of any one of claims 1-15, wherein the cation is sodium cation.

17. The composition of any one of claims 1-15, wherein the cation is potassium cation.

18. The composition of any one of claims 1-17, wherein the molecule has the chemical formula C_{(MC)}H_{(MH)}X_{(MX)}N_{(MN)}O_{(MO)}S_{(MS)}; the anion has the chemical formula C_{(AC)}H_{(AH)}X_{(AX)}N_{(AN)}O_{(AO)}S_{(AS)}¹⁻; MC is an integer from 6 to 32; MH is an integer from 6 to 50; MH is both at least half MC and no more than 1.75 times greater than MC; X is a halogen selected from F, Cl, Br, and I; MX is either 0, 1, or 2; MN is either 0, 1, or 2; MO is an integer from 1 to 12; MC is at least 2 times greater than MO; MS is either 0 or 1; AC equals MC; AH equals MH minus 1; AX equals MX; AN equals MN; AO equals MO; and AS equals MS.

19. The composition of any one of claims 1-18, for use as a medicament.

20. A method to administer a bioactive agent to a subject, comprising providing a composition according to any one of claims 1-19, and administering the composition to the subject.

21. The method of claim 20, wherein the composition is formulated for oral administration, and the composition is formulated to allow the conversion of an anionic form of the bioactive agent into a molecular form of the bioactive agent before the bioactive agent reaches the stomach of the subject to allow absorption of the bioactive agent by the epithelial lining of the gastrointestinal tract between the lips and the stomach, excluding the stomach and the outer surfaces of the lips, and including the esophagus and the inner surfaces of the lips.
